(19) United States
(12) Patent Application Publication
(10) Pub. No.: US 2003/0109741 A1
(43) Pub. Date: Jun. 12, 2003

(54) Mixtures of semi-esters of polybasic organic acids and long-chain alkanols, the production and the use thereof

(76) Inventors: Peter Weyland, Frankenthal (DE); Susanne Demharter, Mannheim (DE); Peter Danisch, Ludwigshafen (DE); Gunter Oetter, Frankenthal (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/221,721

(22) PCT Filed: Mar. 15, 2001
(86) PCT No.: PCT/EP01/02952

(30) Foreign Application Priority Data
Mar. 16, 2000 (DE) 100 12 722.3

(51) Int. Cl. C07C 69/74

(52) U.S. Cl. 560/1

(57) ABSTRACT

Described are complex mixtures of partial esters of polybasic organic acids and long-chain alkanols, alkoxyalkanols and diols, especially thick oils from the oxo process, their preparation and their use as inexpensive high-performance emulsifiers and especially as assistants in leather manufacture.

This invention relates to complex mixtures of partial esters of polybasic organic acids and long-chain alkanols, alkoxyalkanols and diols, especially thick oils from the oxo process, their preparation and their use as inexpensive high-performance emulsifiers and especially as assistants in leather manufacture.

Leather manufacture, as well as the actual organic and/or inorganic tanning agents, utilizes numerous assistant chemicals to substantially influence the properties of the leather, especially its suppleness, hand and performance characteristics, especially its water absorbency and water permeability.

A significant group of assistants is that of the fatliquoring agents which, embedded in the leather, are intended to prevent harshening of the leather and in specific embodiments to act as hydrophobicizers and improve the water resistance of the leather. The effect due to fatliquoring or hydrophobicizing agents should be very durable and withstand even treatments of the finished leather with water, aqueous surfactant solutions and also, if appropriate, drycleaning agents.

It follows that a fatliquoring or hydrophobicizing agent has to be capable of penetrating deep into the microstructure of the leather, to proof and soften it and to become attatched therein in a manner resistant to laundering and dry cleaning if it is perform its function adequately. A further, essential requirement is that the finished leather materials shall not have any greasy surface.

DE 16 69 347 discloses the fatliquoring of leather with water-emulsifiable sulfosuccinic partial esters. However, the leathers thus treated are not waterproof.

EP-A-1 93 832 discloses preparing waterproof leathers by treating the leather with a combination of water-emulsifiable sulfosuccinic partial esters with impregnating and/or hydrophobicizing fatliquors and subsequent fixation with aluminum, chromium or zirconium salts.

EP-A-372 746 describes leather fatliquors comprising copolymers formed from a predominant amount of hydrophobic monomers and a minor amount of hydrophilic monomers and EP-A-412 398 describes leather fatliquors comprising carboxyl-containing copolymers.

DE-A-41 29 244 describes a fatliquoring process utilizing a similar principle. The active substance comprises aqueous dispersions of oligomers having carboxyl groups and ester groups with or without polyether chains.

DE-A-196 44 242 recommends the use of reaction products of long-chain aliphatic monocarboxylic acids, for example stearic acid, with low molecular weight aliphatic hydroxypolycarboxylic acids, for example citric acid, and of reaction products of fatty alcohols, for example stearyl alcohol, with pyromellitic dianhydride in a molar ratio of about 1:2 as leather fatliquors. The solid reaction products are converted into creamy pastes using emulsifiers. They do provide good water resistance, but their pasty consistency makes them inconvenient to apply. A further disadvantage is that they have to be prepared from relatively costly starting materials.

DE-A-44 05 205 discloses the use of water-dispersible partial esters of polybasic, preferably ter- or tetrabasic, cyclic carboxylic acids with monofunctional fatty alcohols of exactly defined chain length for leather fatliquoring. Illustrative is a partial ester prepared from pyromellitic dianhydride and a saturated C18 fatty alcohol in a molar ratio of 1:2.

Important properties of these agents are said to be, on the one hand, the secure fixing of the amphiphilic substances via the free carboxyl groups and, on the other, the exact tunability of the penetration into the leather without forming a greasy surface. However, to provide these properties, the molecular weight of the partial esters has to be very precisely controlled in this method, necessitating the use of disproportionately costly fatty alcohols of defined chain length and structure. It is additionally recommended that the use of these products is followed by a fixing step in the form of a metal salt treatment, especially chrome tanning. The products used in this reference are generally present as creamy or solid dispersions, which is not exactly helpful to the user.

The oxo process mentioned at the outset is a widely practiced industrial process for producing aldehydes and alcohols. In this process, carbon monoxide and hydrogen are added to the double bond of olefins in the presence of suitable catalysts. The oxo process is useful for producing a very wide spectrum of industrially useful aliphatic substances having carbon chains of widely differing lengths. Because of its immense industrial importance, the oxo process has been developed and modified in many directions. A good overview of the technology of the oxo process is found for example in B. Cornils, in J. Falbe, New Syntheses with Carbon-Monoxide, Springer-Verlag, Berlin (1980), Ullmanns Encyklopädie der technischen Chemie, 4th Edition, Volume 7, pages 118 ff., and Winnacker-Küchler, 4th Edition, Volume 5, pages 537 ff.

An important embodiment, using phosphine-modified cobalt catalysts, is a direct way of obtaining useful long-chain alcohols in high yields (Winnacker-Küchler, 4th Edition, Volume 5, pages 537 ff.).

As with any industrial process, the oxo process by-produces products which, because of parallel and subsequent reactions, in an embodiment of the process that is directed to the production of long-chain alcohols, are substantially a mixture of relatively high molecular weight alcohols, ethers, esters and diols.

This mixture, frequently referred to as “thick oil” in the literature, is obtained as the bottom product of the distillation of the crude oxo product. This thick oil, hereinafter referred to as oxo thick oil in order to distinguish if unambiguously from other thick oils, is at present valued only according to its calorific value and it thus constitutes a charge on the oxo process.

It has now been found that, surprisingly, complex mixtures of amphiphilic partial esters of polybasic acids and relatively high molecular weight alkanols useful as emulsifiers, especially for leather fatliquors and hydrophobicizers, are economical to produce using oxo thick oils.

The present invention accordingly provides not only the complex mixtures of amphiphilic partial esters of polybasic acids and relatively high molecular weight alkanols that are extremley useful as emulsifiers, especially for leather fatliquors and hydrophobicizers, and preferably those where the alcohol components are constituents of oxo thick oils, but also their production, formulations of these partial ester mixtures and their use as emulsifiers for fatliquoring and hydrophobicizing compositions.

From a first aspect the present invention provides mixtures of monoesters of di- or tribasic carboxylic acids of the formulae I and II

(MOOC)a-R1-CO—OR2 (I)

[(MOOC)a-R1-CO—O]2R3 (II)

where a is 1 or 2, M is hydrogen or one metal equivalent,

R1 is a di- or trivalent saturated or mono- or diunsaturated aliphatic or cycloaliphatic hydrocarbon radical of 2 to 6 carbon atoms with or without sulfonic acid group substitution or is a di- or trivalent aromatic hydrocarbon radical of 6 carbon atoms,

R2 is predominantly branched unsubstituted or hydroxyl-, alkoxy-, alkylcarbonyloxy- or alkoxycarbonyl-substituted alkyl of 9 to 51 carbon atoms, preferably 9 to 45 carbon atoms, and

R3 is alkanediyl of 10 to 30 carbon atoms,

with OH-free ethers and esters of 18 to 45 carbon atoms and optionally alkanols of the formula R2OH and alkanediols of the formula R3(OH)2, the fraction of —OR2 groups being up to 85%, the fraction of (—O)2R3 groups being up to 16% and the fraction of OH-free ethers and esters being up to 45%, based on the sum total SUG of the weights of the OH-free ethers, OH-free esters, alkanols, alkanediols and —OR2- and (—O)2R3 groups present in the mixture.

It is to be noted that the above-specified fraction of the —OR2 groups and the (—O)2R3 groups comprehends all such groups, regardless of the mixture constituent in which they are present, i.e. not only the groups contained in the esters of the formulae I and II but also the —OR2 groups and (—O)2R3 groups contained in any alkanols and alkanediols present.

It is similarly a condition for the reference variable SUG that it is likewise calculated taking into account the weight of all —OR2 groups and (—O)2R3 groups, regardless of the mixture constituent in which they are present.

In a preferred embodiment of the invention, the fraction of alkanols of the formula R2OH and alkanediols of the formula R3(OH)2, based on the total weight of the mixture, is below 5% by weight, preferably below 2% by weight, especially below 0.5% by weight.

In a further preferred embodiment of the invention, the fraction of alkanols of the formula R2OH and alkanediols of the formula R3(OH)2, based on the total weight of the mixture, is up to 65% by weight, preferably up to 60% by weight, especially up to 55% by weight.

Preference is given to inventive mixtures of monoesters of di- or tribasic carboxylic acids of the formulae I and II

(MOOC)a-R1-CO—OR2 (I)

[(MOOC)a-R1-CO—O]2R3 (II)

where a is 1 or 2, M is hydrogen or one metal equivalent,

R1 is a divalent saturated aliphatic hydrocarbon radical of 2 to 4 carbon atoms with or without sulfonic acid group substitution

or is a divalent monounsaturated aliphatic hydrocarbon radical of 2 to 4 carbon atoms,

or is a divalent or trivalent saturated cycloaliphatic hydrocarbon radical of 6 carbon atoms with or without sulfonic acid group substitution

or is a di- or trivalent cycloaliphatic hydrocarbon radical of 6 carbon atoms having one or if appropriate two double bonds

or is a di- or trivalent aromatic hydrocarbon radical of 6 carbon atoms,

R2 comprises the radicals R2A, R2B, R2C, R2D and R2E, where

R2A denotes 1-alkyl and 2-alkyl radicals of 9 to 15 carbon atoms having an average molecular weight of MA

R2B denotes 2-alkyl-1-alkyl radicals of 18 to 30 carbon atoms having an average molecular weight of MB

R2C denotes x-alkyl-y-alkyl radicals of 18 to 30 carbon atoms having an average molecular weight of MC

R2D denotes 1-alkoxyalkyl radicals of 18 to 30 carbon atoms having an average molecular weight of MD

R2E denotes x-alkylcarbonyloxy-y-alkyl radicals and/or x-alkoxycarbonyl-y-alkyl radicals of 27 to 51 carbon atoms having an average molecular weight of ME,

R3 comprises the radicals R3F and R3G, where

R3F denotes 1,2-alkanediyl and/or 2-alkyl-1,3-alkanediyl of 10 to 16 carbon atoms having an average molecular weight of MF

R3G denotes 1,3-alkylalkanediyl of 18 to 30 carbon atoms having an average molecular weight of MG,

with OH-free ethers and esters of 18 to 45 carbon atoms and optionally alkanols of the formula R20H and alkanediols of the formula R3(OH)2,

the —OR2A groups having a fraction of A[%]=0 to 20% by weight,

the —OR2B groups having a fraction of B[%]=0 to 40% by weight,

the —OR2C groups having a fraction of C[%]=0 to 10% by weight,

the —OR2D groups having a fraction of D[%]=0 to 20% by weight,

the —OR2E groups having a fraction of E[%]=0 to 25% by weight,

the (—O)2R3F groups having a fraction of F[%]=0 to 7.5% by weight,

the (—O)2R3G groups having a fraction of G[%]=0 to 8% by weight,

and the OH-free ethers and esters having a fraction of H[%}=0 to 45% by weight, based on the sum total SUG of the weights of the OH-free ethers and esters, alkanols, alkanediols and —OR2 and (—O)2R3 groups present in the mixture.

The values of x and y follow from the hereinbelow described structural types for the constituents of the radicals R2 and R3. Preferably x is in the range from 9 to 15 and y in the range from 10 to 16.

Preference is given to those partial ester mixtures according to the invention wherein the fractions of structures —OR2A, —OR2B, —OR2C, —OR2D, —OR2E, —OR3FO— and —OR3GO— present in the building groups —OR2 and —OR3 and the fraction of ethers and esters are selected in such a way within the framework of the above-indicated limits that the number OHZ defined by equation (GL1)

$$OHZ = 561 \cdot ( \frac{A [ \% ]}{MA} + \frac{B [ \% ]}{MB} + \frac{C [ \% ]}{MC} + \frac{D [ \% ]}{MD} + \frac{E [ \% ]}{ME} ) + 1120 \cdot ( \frac{F [ \% ]}{MF} + \frac{G [ \% ]}{MG} ) \qquad ( GL1 )$$

where A[%], B[%], C[%], D[%], E[%], F[%] and G[%] are the abovementioned percentages of the structures present in the building groups —OR2 and —OR3, based on the sum total weight SUG, and MA, MB, MC, MD, ME, MF and MG are the molecular weights of said structures, is in the range from 65 to 160, preferably in the range from 90 to 140, especially in the range from 90 to 120.

The molecular weights to be used are the number average molecular weights resulting from the formulae of the radicals —OR2 and —OR3. The percentages A[%], B[%], C[%], D[%], E[%], F[%] and G[%] of the mixture constituents shall add up to (100-H)[%].

Preference is further given to those inventive mixtures of monoesters of the formulae I and II wherein

R1 is ethane-1,2-diyl, sulfoethane-1,2-diyl, ethene-1,2-diyl, propane-1,2-diyl, propane-1,3-diyl, sulfopropane-1,2-diyl, sulfopropane-1,3-diyl, propene-1,2-diyl, cyclohexane-1,2-diyl, sulfocyclohexane-1,2-diyl, disulfocyclohexane-1,2-diyl, cyclohex-1-,-2-,-3- or -4-ene-1,2-diyl, cyclohex-1,3-,-1,4-,-2,4- or -2,5-diene-1,2-diyl, cyclohexane-1,2,4-triyl, sulfocyclohexane-1,2,4-triyl, disulfocyclohexane-1,2,4-triyl, cyclohex-1-,-2-,-3- or 4-ene-1,2,⁴-triyl, cyclohex-1,3-,-1,4-,-2,4- or -2,5-diene-1,2,4-triyl, phenylene-1,2,3- or -4-sulfophenylene-1,2- and phenyl-1,2,4-triyl and especially those in which

R1 is ethene-1,2-diyl, sulfoethane-1,2-diyl, 1,2-phenylene or 3- or 4-sulfo-1,2-phenylene or phenyl -1,2,4-triyl.

R1 radicals bearing sulfone groups are particularly preferred.

The monoester mixtures according to the invention may also contain components that differ from each other with regard to the acid radicals R1.

Preference is also given to those inventive mixtures of monoesters of the formulae I and II in which the composition of R2 and R3 is defined in such a way that

A[%] is from 4 to 15, especially from 5 to 10,

B[%] is from 20 to 35, especially from 25 to 31,

C[%] is from 1 to 5, especially from 2 to 4,

D[%] is from 3 to 20, especially from 5 to 15,

E[%] is from 5 to 25, especially from 10 to 20,

F[%] is from 0.8 to 5, especially from 1 to 4,

G[%] is from 1 to 5, especially from 2 to 4, and

H[%] is from 10 to 40, especially from 20 to 35.

In the following further particulars concerning the structure of the —R2 and —R3 radicals, R and R′ are identical or different, preferably linear, alkyl radicals of 7 to 13 carbon atoms.

The R2A radicals are preferably linear 1-alkyl or 2-methyl-1-alkyl radicals derived respectively from alkanols and methylalkanols (oxo alcohols) of the structural type R—CH2CH2-OH or R—CH(CH3)-OH, the fraction of unbranched alkanols generally being predominant.

The —R2B 2-alkyl-1-alkyl radicals are preferably derived from branched alkanols HO—R2B of the structural type R—CH2-CH2-CH(CH2OH)—R′, which may be formed from aldehydes of the formulae R—CH2-CHO and R′—CH2-CHO by aldol condensation and subsequent water elimination and hydrogenation.

The x-alkyl-y-alkyl radicals —R2C are derived from secondary alkanols HO—R2C of the structural type R—CH2-CH(OH)—CH(CH3)-R′. These are probably formed by aldol condensation of aldehydes of the formulae R—CH2-CHO and R′—CH2-CHO and subsequent hydrogenation of the aldehyde group to the methyl group. The alkoxyalkyl radicals —R2D are derived from alkanols HO—R2D of the structural types R—CH2-CH(CH20H)—OCH2-R′ and R—CH(CH20H)CH2-OCH2-R′. These can be formed by acetalization of R—CH2-CHO with 2 mol of R′—CH2-CH2OH, elimination of one mole of the alkanol to form vinyl ethers of the formula III

R—CH═CH—O—CH2-CH2-R′ (III)

and hydroformylation of the vinyl ethers.

The —R2E radicals bearing ester groups are derived from one or more alkanols HO—R2E of the structural types

R—CH2-CH(OH)—CH(CH2-OCO—CH2-R)—R′

R—CH2-CH(OH)—CH(CH2-OCO—CH2-R′)—R′

R—CH2-CH(OH)—CH(COO—C2H4-R)—R′

R—CH2-CH(OH)—CH(COO—C2H4-R′)—R′

R—CH2-CH(OCO—CH2-R)—CH(CH20H)—R′ and

R—CH2-CH(OCO—CH2-R′)—CH(CH20H)—R′.

These can be formed by a Cannizzaro reaction (disproportionation) of 1 mol of one of the abovementioned aldols with one mole of an aldehyde R—CH2-CHO or R′—CH2-CHO and subsequent esterification of the resulting carboxylic acids and alkanols.

The alkanediyl radicals —R3F are derived from diols (HO)2-R3F of the structural types R—CH2-CH(CH2OH)—OH and R—CH(CH20H)—CH2OH. These can be formed by elimination of one mole of olefin from the alkoxy groups of the abovementioned alkoxyalkanols.

Alkylalkanediyl radicals —R3G are derived from diols (HO)2-R3G of the structural type R—CH2-CH(OH)—CH(CH2OH)—R′, which are obtainable by hydrogenation of the abovementioned aldols.

The OH-free ethers conform to the structure R—CH2CH2-O—CH2-R′. They are obtained on hydrogenating the vinyl ethers of the formula III.

The OH-free esters can be linear or branched. The linear esters conform to the structural type R—CH2CH2-OCO—CH2R′, and are formed by Cannizzaro disproportionation of aldehydes of the formula R—CH2-CHO and R′—CH2-CHO and subsequent esterification of the resulting carboxylic acids and alkanols.

The branched esters conform to the structural types

R—CH2-CH2-CH(CH2-OCO—CH2-R)—R′

R—CH2-CH2-CH(CH2-OCO—CH2-R′)—R′

R—CH2-CH2-CH(COO—C2H4-R)—R′ and

R—CH2-CH2-CH(COO—C2H4-R′)—R′

R—CH2-CH(OCO—CH2-R)—CH(CH3)-R′ and

R—CH2-CH(OCO—CH2-R′)—CH(CH3)-R′.

They can be formed by water elimination and subsequent hydrogenation of the abovementioned alkanols bearing ester groups.

The present invention further provides a process for preparing the above-described mixtures of monoesters of the formulae I and II by reacting inner anhydrides of di- or tribasic carboxylic acids with alkanols of medium and/or relatively large chain length, which comprises

A) reacting inner anhydrides of the formula IV

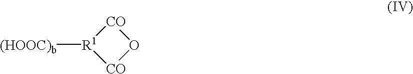

where b is 0 or 1,

R1 is a di- or trivalent saturated or mono- or diunsaturated aliphatic or cycloaliphatic hydrocarbon radical of 2 to 6 carbon atoms or a di- or trivalent aromatic hydrocarbon radical of 6 carbon atoms, with or without a sulfo group,

at from about 20 to not more than 120° C. and preferably at from 75 to 100° C. in an equivalent ratio of from 1 to 1 to 1 to 5 with a mixture of alcohols of the formula R2OH, where

R2 is predominantly branched, optionally hydroxyl-, alkoxy-, alkylcarbonyloxy- or alkoxycarbonyl-substituted alkyl of 9 to 51 carbon atoms, preferably 9 to 45 carbon atoms, alkanediols of the formula (HO)2R3, where

R3 is alkanediyl of 10 to 30 carbon atoms, and OH-free ethers and esters of 18 to 45 carbon atoms, and

B) to prepare mixtures according to the invention in which R1 has a sulfo group from a sulfo-free anhydride (IV) conducting the preparation step A) using an anhydride (IV) whose R1 radical is at least monounsaturated and then reacting the monoester mixture obtained according to section A), if appropriate after at least partial neutralization of its free carboxyl groups, in a conventional manner with a water-soluble sulfite, bisulfite or disulfite to add the sulfite or bisulfite to the double bond of the R1 group to form the desired sulfonic acid.

The amounts of the reactants to be reacted with one another in preparation step A) depend on the molecular weight of the anhydrides of formula IV and on the equivalent weight of the mixture of the alcohols HOR2 and diols (HO)2R3, which is obtained from the OH number thereof in a known manner.

The reaction of the di- or tricarboxylic anhydrides of the formula IV with the mixtures of OH-containing compounds can be carried out with or without solvent or diluent. When the anhydrides are reacted with the alcohol and diol mixture in an equivalent ratio below 1—that is, when the alcohol and diol components are present in excess—this excess can serve as a solution and dilution medium for the reaction. Generally, preparation step A) will provide adequate conversion of the reactants within from 2 to 5 hours under the stated reaction conditions.

The process of the invention can be carried out using one or more different inner anhydrides of the formula IV.

When, as part of the process according to the invention, a sulfonation is to be carried out by sulfite or bisulfite addition as per the optional process step B), then the reaction in process step A) between the inner anhydride IV and the alcohol and diol components is advantageously carried out in an equivalent ratio above 0.5, i.e. in the range from 1:1 to 1:2, preferably in the range from 1:1 to 1:1.2 and especially in the range from 1:1 to 1:1.05.

Preferred monoester mixtures according to the invention are obtained on using inner anhydrides of the formula IV where

R1 is a divalent saturated aliphatic hydrocarbon radical of 2 to 4 carbon atoms with or without sulfonic acid group substitution

or is a divalent monounsaturated aliphatic hydrocarbon radical of 2 to 4 carbon atoms,

or is a divalent or trivalent saturated cycloaliphatic hydrocarbon radical of 6 carbon atoms with or without sulfonic acid group substitution

or is a di- or trivalent cycloaliphatic hydrocarbon radical of 6 carbon atoms having one or if appropriate two double bonds

or is a di- or trivalent aromatic hydrocarbon radical of 6 carbon atoms.

Preferred monoester mixtures according to the invention are particularly usefully prepared using anhydrides of the formula IV in which

R1 is ethane-1,2-diyl, sulfoethane-1,2-diyl, ethene-1,2-diyl, propane-1,2-diyl, propane-1,3-diyl, sulfopropane-1,2-diyl, sulfopropane-1,3-diyl, propene-1,2-diyl, cyclohexane-1,2-diyl, sulfocyclohexane-1,2-diyl, disulfocyclohexane-1,2-diyl, cyclohex-1-,-2-,-3- or -4-ene-1,2-diyl, cyclohex-1,3-,-1,4-,-2,4- or -2,5-diene-1,2-diyl, cyclohexane-1,2,4-triyl, sulfocyclohexane-1,2,4-triyl, disulfocyclohexane-1,2,4-triyl, cyclohex-1--2-, -3- or -4-ene-1,2,4-triyl, cyclohex-1,3-, -1,4-,-2,4- or -2,5-diene-1,2,4-triyl, phenylene-1,2,3- or 4-sulfophenylene-1,2- and phenyl-1,2,4-triyl and especially those in which

R1 is ethene-1,2-diyl, sulfoethane-1,2-diyl, 1,2-phenylene, 3- or 4-sulfo-1,2-phenylene and phenyl -1,2,4-triyl.

Useful di- or tricarboxylic anhydrides of the formula IV include for example succinic anhydride, maleic anhydride, glutaric anhydride, methylsuccinic anhydride, citraconic anhydride, hexahydrophthalic anhydride (cyclohexanedicarboxylic anhydride), all isomeric dihydrophthalic anhyrides, all isomeric tetrahydrophthalic anhydrides, phthalic anhydride and trimellitic anhydride, of which maleic anhydride, phthalic anhydride and trimellitic anhydride are particularly preferred.

The process of the invention preferably uses mixtures of alcohols R2OH and diols R3(OH)2 comprising

from 0 to 20% by weight and preferably from 4 to 15% by weight of C9-C15 alkanols

from 0 to 40% by weight and preferably from 20 to 35% by weight of C18-C30 2-alkyl alcohols

from 0 to 10% by weight and preferably from 1 to 5% by weight of C18-C30 secondary alcohols

from 0 to 20% by weight and preferably from 3 to 20% by weight of C18-C30 ether alcohols

from 0 to 25% by weight and preferably from 5 to 25% by weight of C27-C51 ester alcohols

from 0 to 7.5% by weight and preferably from 0.8 to 5% by weight of C10-C16 1,2-diols

from 0 to 8% by weight and preferably from 1 to 5% by weight of C18-C30 diols and

from 0 to 45% by weight and preferably from 10 to 40% by weight of OH-free C18-C45 ethers and esters.

The aforementioned mixture constituents R2OH and R3(OH)2 may themselves be mixtures of compounds of various structural types as hereinbelow illustrated in Table 1.

TABLE 1

| | | | Preferred |
|---|---|---|---|
| | | Fraction | fraction |
| | | range | range |
| | | [% by | [% by |
| Designation | Structural type | weight] | weight] |
| Oxo alcohols | R—CH2—OH or R—CH(CH3)— | 0 to 20 | 4 to 15 |
| C9-C15 | OH | | |
| 2-Alkyl | R—CH2—CH2—CH(CH2OH)— | 0 to 40 | 20 to 35 |
| alcohols | R′ | | |
| C18-C30 | | | |
| sec. Alcohols | R—CH2—CH(OH)—CH(CH3)— | 0 to 10 | 1 to 5 |
| C18-C30 | R′ | | |
| Ether alcohols | R—CH2—CH(CH2OH)— | 0 to 20 | 3 to 20 |
| C18-C30 | OCH2—R′ and R— | | |
| | CH(CH2OH)CH2—OCH2—R′ | | |
| Ester alcohols | R—CH2—CH(OH)—CH(CH2— | 0 to 25 | 5 to 25 |
| C27-C51 | OCO—CH2—R)—R′, | | |
| | R—CH2—CH(OH)—CH(CH2— | | |
| | OCO—CH2—R′)—R′, | | |
| | R—CH2—CH(OH)—CH(COO— | | |
| | C2H4—R)—R′ | | |
| | R—CH2—CH(OH)—CH(COO— | | |
| | C2H4—R′)—R′ | | |
| | R—CH2—CH(OCO—CH2— | | |
| | R)—CH(CH2OH)—R′ | | |
| | R—CH2—CH(OCO—CH2— | | |
| | R′)—CH(CH2OH)—R′ | | |
| 1,2- and | R—CH2—CH(CH2OH)—OH and | 0 to 7.5 | 0.8 to 5 |
| 1,3-Diols | R—CH(CH2OH)—CH2OH | | |
| C10-C16 | | | |
| Alkyl-1,3-diols | R—CH2—CH(OH)— | 0 to 8 | 1 to 5 |
| C18-C30 | CH(CH2OH)—R′ | | |
| Ethers (OH- | R—CH2CH2—O—CH2—R′ | 0 to 45 | 10 to 40 |
| free) | | | |
| C18-C45 | | | |
| Esters | R—CH2CH2—O—CH2—R′ | | |
| (OH-free) | R—CH2—CH2—CH(CH2— | | |
| C18-C45 | OCO—CH2—R)—R′, | | |
| | R—CH2—CH2—CH(CH2— | | |
| | OCO—CH2—R′)—R′, | | |
| | R—CH2—CH2—CH(COO— | | |
| | C2H4—R)—R′, | | |
| | R—CH2—CH2—CH(COO— | | |
| | C2H4—R′)—R′, | | |
| | R—CH2—CH(OCO—CH2— | | |
| | R)—CH(CH3)—R′, | | |
| | R—CH2—CH(OCO—CH2— | | |
| | R′)—CH(CH3)—R′, | | |
| | R—CH2—CH(OCO—CH2— | | |
| | R)—CH(CH3)—R′, | | |
| | R—CH2—CH(OCO—CH2— | | |
| | R′)—CH(CH3)—R′ | | |

The mixtures of the stated composition which are to be used for the process are obtainable by mixing a selection made from the stated group of substances, the amounts being determined so that the above-indicated upper limits for the fractions of the mixture constituents are not exceeded and the % fractions of the mixed components add up to 100%.

Preferably the components and their fractions in the mixture are selected so that the mixture has an OH number between 65 and 160, especially between 90 and 140 mg of KOH/g.

Equivalence ratios of about 1:1 for the reaction of the inner anhydride IV with the alkanol and diol mixture in the process of the invention provides specific monoester mixtures according to the invention which generally include less than 5% by weight, preferably less than 2% by weight and especially less than 0.5% by weight of alkanols and diols.

Reacting the inner anhydride IV with the alkanol and diol mixture in equivalence ratios of below 1, for example in an equivalence ratio of 1:2 or 1:3, provides specific mixtures which include higher fractions of alkanols and diols, for example about 37% by weight and 55% by weight respectively, and which are particularly readily processable into ready-to-use leather fatliquoring and hydrophobicizing formulations, since they are easy to mix with further fatliquoring and hydrophobicizing agents and assistants and since the excess alkanol and diol components likewise provide a good fatliquoring effect.

A very particular advantage of the present invention is that the monoester mixtures of the invention are obtainable in a very simple manner by reacting the inner anhydrides of the formula IV with oxo thick oils as an alcohol component.

The use of these oxo thick oils not only provides the mixtures of monoesters of polybasic carboxylic acids of the invention in a particularly economical manner, but also opens up a sensible recovery route for the oxo thick oils, which otherwise are considered by-product and waste. The use of oxo thick oils in the production processes of the invention constitutes a particularly preferred embodiment thereof, and similarly the thus produced monoester mixtures of the invention constitute a particularly preferred embodiment of the present invention.

Useful oxo thick oils for the purposes of the present invention comprise

from 0 to 20% by weight, preferably from 4 to 15% by weight, of C9-C15 oxo alcohols

from 0 to 40% by weight, preferably from 20 to 35% by weight, of C18-C30 2-alkyl alcohols

from 0 to 10% by weight, preferably from 1 to 5% by weight, of C18-C30 sec. alcohols

from 0 to 20% by weight, preferably from 3 to 20% by weight, of C18-C30 ether alcohols

from 0 to 25% by weight, preferably from 5 to 25% by weight, of C27-C51 ester alcohols

from 0 to 7.5% by weight, preferably from 0.8 to 5% by weight, of C10-C16 1,2-diols

from 0 to 8% by weight, preferably from 1 to 5% by weight, of C18-C30 diols

from 0 to 45% by weight, preferably from 10 to 40% by weight, of OH-free C18-C45 ethers and esters,

and have an OH number in the range from 65 to 160, preferably from 90 to 140. To select useful oxo thick oils it is merely necessary to analyze the available products as to whether they have the above-described composition needed.

The qualitative analysis of oxo thick oils and the quantitative determination of the main constituents thereof may be effected in a conventional manner (cf. for example EP-A-0 718 351) by gas-chromatographic separation with in-line mass spectroscopy of the fractions obtained.

It is advantageous to use 25 m or 50 m SE 54 fused silica capillaries. Illustrations of the resulting gas chromatograms are to be found on pages 14 and 15 of above-cited EP-A-0 718 351. By measuring the peak areas of the main components and normalizing to 100% it is possible to determine the composition of the oxo thick oils with sufficient accuracy for the purposes of the present invention.

Oxo thick oils specifically, but certainly not exclusively, useful for preparing the monoesters of the invention are “oxo oil 911”, “oxo oil 13” and “oxo oil 135”.

The monoester mixtures of the invention may be rendered water-soluble or water-dispersible by conversion of some at least of their free carboxylic acid groups present into metal salts (M in formula I and II at least partly=metal atom), preferably into alkali metal salts, especially into sodium or potassium salts, i.e. by complete or partial neutralization of the carboxylic acid groups, and then be used as very effective and very economically produced dispersants or emulsifiers for stable fine dispersion of water-insoluble substances in an aqueous phase. The use of the monoester mixtures according to the invention as emulsifiers likewise forms part of the subject matter of the present invention.

A particularly advantageous use, which likewise forms part of the subject matter of this invention, comprises using the monoester mixtures according to the invention as assistants in leather making. In this use, the monoester mixtures according to the invention are used in combination with leather fatliquoring agents and/or leather hydrophobicizing agents.

Useful fatliquoring and/or hydrophobicizing agents include in particular unesterified oxo thick oils. In addition to these or instead of them, however, it is also possible to use further substances, for example white oil, paraffins, native oils and/or silicones as fatliquoring and/or hydrophobicizing agents.

Particularly good hydrophobicizing effects are obtained on combining the emulsifiers of the invention with unesterified oxo thick oils and/or optionally further fatliquoring and/or hydrophobicizing substances. Useful silicones for this purpose include in particular known polysiloxanes bearing carboxyl groups attached via bridge members to a linear or branched siloxane backbone. These compounds preferably conform to the formula V

[siloxane backbone-][-BR(—COOH)p]y, (V)

where BR is a (p+1) valent organic bridge member attached to a silicon atom on the backbone, p is from 1 to 10 and y is selected such that the compound contains from 0.01 to 2.0, preferably from 0.02 to 1.5, meq/g of carboxyl groups.

Useful BR bridge members conform for example to the formula (VI)

-Z-(A-)p (VI)

where A is a divalent aliphatic straight-chain or branched hydrocarbon radical, a divalent, cyclic or bicyclic, saturated or unsaturated hydrocarbon radical or a divalent aromatic hydrocarbon radical, Z is a direct bond, an oxygen atom or a group of the formula —NR4-, —CO—, or —CO—O— or a (p+1) valent organic radical of the formula VIa

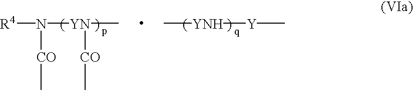

where p and q are independently from 0 to 10 and the sum p+q is likewise in the range from 0 to 10 and R4 in the building groups mentioned is hydrogen or C1- to C4-alkyl and Y denotes identical or different straight-chain or branched alkanediyl radicals of 2 to 4 carbon atoms.

Examples of suitable polysiloxanes having a linear siloxane backbone are those of the formula VII

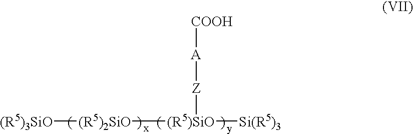

where the R3 radicals are the same or different and independently represent hydrogen, hydroxyl, C1- to C4-alkyl, phenyl, C1- to C4-alkoxy, amino, mono-C1- to C4-alkylamino, di-C1- to C4-alkylamino, chlorine or fluorine, although one of each R5 radical at the chain ends may also be -Z-A-COOH,

A is a linear or branched C5- C25-alkylene group,

Z is a direct bond, an oxygen atom or a group of the formula —NR4-, —CO—, —CO—NR4-, or —CO—O—, where R4 is hydrogen or C1- to C4-alkyl, and

the indices x and y of the associated randomly distributed structural units sum to a total in the range from 50 to 500, a molecule VII containing on average from 1 to 50, preferably from 2 to 20 and especially from 2.5 to 15 carboxyl groups.

Siloxanes which are particularly useful for combination with the monoesters according to the invention have the formula VII where the x+y sum is in the range from 100 to 300 and especially in the range from 120 to 200 and the x:y ratio is in the range from 99:1 to 9:1, and also the formula VII where R5 is C1- to C3-alkyl and especially methyl.

Siloxanes of the formula VII are known from EP-B-0 745 141.

Examples of siloxanes having a linear or branched backbone include the WO-98/21369 compounds of the formula VIII

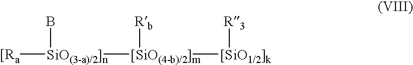

in which R, R′ and R″, each independently, are C1- to C6-alkyl or phenyl or a polysiloxane radical of the formula VIIIa

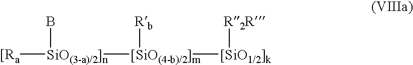

where, in the formula VIII, R′ and R″, each independently, are C1- to C6-alkyl or phenyl and R and R′″, each independently, are C1- to C6-alkyl, C1- to C6-alkoxy, OH or phenyl, and

0≦a≦2, 1≦b≦3, 1≦n≦60,20≦m≦800, and 0≦k≦(2−b)m+[(1−a)n+2],

and B is a radical of the formula IX

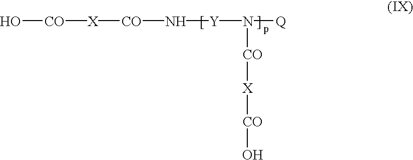

in which p is from 0 to 10, Y and Q, each independently, are alkanediyl of short or medium chain length and X is a divalent aliphatic hydrocarbon radical which is saturated or unsaturated and is straight-chain or branched, a divalent cyclic or bicyclic hydrocarbon radical which is saturated or unsaturated or a divalent aromatic hydrocarbon radical,

where selectively some of the substituents of the formula IX may be replaced by radicals of the formula X or XI

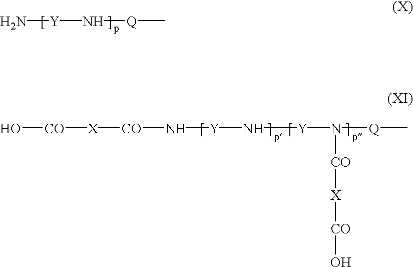

where X, Y, Q and p are each as defined above and

p′+p″=p. The polysiloxanes VIII and VIIIa have a carboxyl group content of from 0.02 to 1.0 meq/g and a molar mass in the range from 2×103 to 60×103 g/mol.

The preferred use for leather assistants advantageously utilizes aqueous formulations which, as well as the emulsifiers according to the invention, include the substances required for fatliquoring and/or hydrophobicization as an emulsion in water. In principle, these fatliquoring and hydrophobicizing agents, besides the emulsifiers of the invention, require no further amphiphilic components for emulsification; but these further amphiphilic components may be added when specific effects are desired. Useful fatliquoring and/or hydrophobicizing components for these formulations include the abovementioned substances, especially unesterified oxo thick oils. In addition to or in place of unesterified oxo thick oils, however, it is also possible to use further substances, for example white oil, paraffins, native oils and/or silicones in the formulations, and formulations that include a fraction of silicones, preferably the siloxanes V mentioned above, in particular siloxanes VII or VIII, provide particularly good hydrophobicizing effects. Such formulations advantageously include from I to 20%, preferably from 2 to 10%, especially from 2.5 to 8%, by weight of the silicone, in particular the siloxane V.

The aqueous formulations mentioned likewise form part of the subject matter of the present invention.

Besides water they generally include, based on the nonvolatile fraction,

from 10 to 40% by weight of the monoesters according to the invention and/or salts thereof,

from 0 to 20% by weight of further emulsifiers

from 0 to 90% by weight of unesterified oxo thick oil

from 0 to 90% by weight of further hydrophobicizing substances of the abovementioned classes,

from 0 to 10% by weight of assistants, such as antifoams, antifreezes, bactericides, fungicides, metal-complexing agents, storage stabilizers, dilution assistants and the like.

The solids content of the aqueous formulations is advantageously in the range from 20 to 60% by weight, but it may, if desired, also be adjusted downward or upward so it may be conformed to particular users, end uses and equipment requirements.

It will be appreciated that it is also possible to prepare, and if required use, anhydrous formulations of the above-indicated composition. These anhydrous formulations therefore also form part of the subject matter of this invention.

The aqueous formulations of the invention constitute relatively low viscosity liquids, have comparatively low emulsifier concentrations, possess very good stability in storage, are highly impervious to water hardness and retanning agents and are readily thinnable to the use concentration required for leather treatment.

The leathers treated therewith do not have a greasy surface but a pleasant hand, a uniform color and are water-impermeable. The effects obtained are very stable to water, aqueous surfactant solutions and dry cleaning agents.

Similarly, the use of the monoesters according to the invention as assistants in leather treatment processes forms part of the subject matter of the present invention. For this use, the monoesters are advantageously used in the form of the above-described formulations, preferably in aqueous formulations. Leather treatment floats generally include from 0.5 to 8%, preferably from 1.5 to 5%, by weight of the nonvolatiles in the formulations according to the invention, based on the shaved weight of the leather (wet blue). Otherwise leather treatment is effected in a conventional manner.

The examples hereinbelow illustrate the invention. The composition of the type of oxo thick oil used therein is discernible from Table 2 hereinbelow:

TABLE 2

| Oxo thick oil type | “Oxo oil 135” |
|---|---|
| Components | Fractions in % by weight |
| Oxo alcohols C9-C15 | 6.1 |
| 2-alkyl alcohols C18-C30 | 27.9 |
| Sec. alcohols C18-C30 | 4.1 |
| Ether alcohols C18-C30 | 6.7 |
| Ester alcohols C27-C45 | about 17.4 |
| 1,2-diols C10-C16 | 1.0 |
| Diols C18-C30 | 2.3 |
| Ethers C18-C45 (OH-free) | 5.4 |
| Esters C18-C45 (OH-free) | about 29.1 |

The qualitative analysis of oxo oils 911 and 135 and the quantitative determination of the constituent fractions reported in Table 2 were effected in the conventional manner by gas-chromatographic separation and in-line mass spectroscopy of the fractions obtained. For oxo oil 911 the separation was carried out using a 50 m SE 54 fused silica capillary. Oxo oil 135 was separated using a corresponding 25 m capillary. The main components reported in the table were determined by normalizing the GC peak areas to 100%.

The oxo oil 135 used in the examples is additionally characterized by an OH number of 117. The OH number was determined along the lines of German Standard Specification DIN 53240 of December 1971 and DIN 53240 Part II of December 1993.

The testing of the leather for water permeability was carried out using a Bally penetrometer as per IUP 10 of the International Union of Leather Technologists and Chemists Societies (cf. Das Leder, Volume 12, pages 26-40 (1961)).

### EXAMPLE 1

A) Preparation of a Monoester According to the Invention

470 g (0.9 mol by OH number) of oxo thick oil type 135 are initially introduced into a 1 000 ml three-neck glass flask equipped with stirrer, thermometer, reflux condenser and moisture seal and are heated to 100° C. in an oil bath. 133.3 g (0.9 mol) of phthalic anhydride are then added with stirring and the mixture is stirred at 100° C. for 7 hours.

The monoester thus obtained is cooled down to 40° C.; the conversion is virtually quantitative.

B) Preparation of a Formulation According to the Invention

200 g of the monoester prepared are adjusted to pH 8 at 40° C. by careful addition of 25% by weight aqueous sodium hydroxide solution with stirring. This is followed by the addition of 340 g of oxo thick oil type 135, 225 g of white oil and 1 200 g of water with stirring and the mixture is converted into an emulsion in a conventional manner, for example using a slot homogenizer.

The thin liquid emulsion includes 10% by weight of the emulsifier according to the invention. It possesses very good stability in storage (stable for more than 60 days at 23° C., 40° C. and 50° C.) and is very easily thinnable with water to use concentration. It is stable to hard water to at least 40° German hardness.

Oxo thick oils 911 and 13 can be converted into partial esters and partial ester formulations in a similar manner.

### EXAMPLE 2

A) Preparation of a Monoester According to the Invention

183 g (0.3 mol by OH number) of oxo thick oil type 135 are initially introduced into a 500 ml three-neck glass flask equipped with stirrer, thermometer, reflux condenser and moisture seal and are heated to 100° C. in an oil bath. 29.4 g (0.3 mol) of maleic anhydride are then added with stirring and the mixture is stirred at 100° C. for 5 hours.

The monoester thus obtained is cooled down to 40° C.; conversion is virtually quantitative.

B) Preparation of a Formulation According to the Invention

200 g of the monoester prepared are converted into an emulsion as described in Example 1.

The thin liquid emulsion includes 10% by weight of the emulsifier according to the invention. It possesses very good stability in storage (stable for more than 60 days at 23° C., 40° C. and 50° C.) and is very easily thinnable with water to use concentration. It is stable to hard water to at least 40° German hardness.

Oxo thick oils 911 and 13 can be converted into partial esters and partial ester formulations in a similar manner.

### EXAMPLE 3

A) Preparation of a Monoester According to the Invention

549 g (0.9 mol by OH number) of oxo thick oil type 135 are initially introduced into a 1 000 ml three-neck glass flask equipped with stirrer, thermometer, reflux condenser and moisture seal and are heated to 100° C. in an oil bath. 29.4 g (0.3 mol) of maleic anhydride are then added with stirring and the mixture is stirred at 100° C. for 3 hours.

The monoester thus obtained is cooled down to 40° C.; conversion is virtually quantitative.

B) Preparation of a Formulation According to the Invention

The mixture of inventive monoester and excess oxo thick oil 135 that is obtained as per section A is adjusted to pH 8 at 40° C. by careful addition of 25% by weight aqueous sodium hydroxide solution with stirring. This is followed by the addition of 225 g of white oil and 1 300 g of water with stirring and the mixture is converted into an emulsion in a conventional manner, for example using a slot homogenizer.

The thin liquid emulsion includes 10% by weight of the emulsifier according to the invention. It possesses very good stability in storage (stable for more than 60 days at 23° C., 40° C. and 50° C.) and is very easily thinnable with water to use concentration. It is stable to hard water to at least 40° German hardness.

Oxo thick oils 911 and 13 can be reacted and converted into formulations in a similar manner.

### EXAMPLE 4

A) Preparation of a Sulfo-containing Monoester According to the Invention

695 g (1.4 mol by OH number) of oxo thick oil type 135 are initially introduced into a 1 000 ml three-neck glass flask equipped with stirrer, thermometer, reflux condenser and moisture seal and are heated to 100° C. in an oil bath. 132.2 g (1.4 mol) of maleic anhydride are then added with stirring and the mixture is stirred at 100° C. for 5 hours.

The monoester thus obtained is cooled down to 40° C., stirred into 965 g of water and partially neutralized by addition of 61.2 g (0.77 mol) of 50% by weight aqueous sodium hydroxide solution, and the mixture obtained is heated to 80° C. 133.1 g of sodium disulfite are then added with stirring. And the batch is further stirred at 80° C. for 6 hours.

B) Preparation of a Formulation According to the Invention

400 g of the 50% by weight mixture of the monoester/sulphite adduct that is obtained as per section A is adjusted to pH 8 at 40° C. by careful addition of 25% by weight aqueous sodium hydroxide solution with stirring. This is followed by the addition of 340 g of oxo thick oil 135, 225 g of white oil and 1 000 g of water with stirring and the mixture is converted into an emulsion in a conventional manner, for example using a slot homogenizer.

The thin liquid emulsion includes 10% by weight of the emulsifier according to the invention. It possesses very good stability in storage (stable for more than 60 days at 23° C., 40° C. and 50° C.) and is very easily thinnable with water to use concentration. It is stable to hard water to at least 40° German hardness.

Oxo thick oils 911 and 13 can be reacted and converted into formulations in a similar manner.

### EXAMPLE 5

A) Preparation of a Sulfo-containing Monoester According to the Invention

The batch described in part A of Example 4 is repeated.

B) Preparation of a Formulation According to the Invention

400 g of the 50% by weight mixture of the monoester/sulphite adduct that is obtained as per section A is adjusted to pH 8 at 40° C. by careful addition of 25% by weight aqueous sodium hydroxide solution with stirring. This is followed by the addition of 340 g of oxo thick oil 135, 225 g of white oil, 80 g of a silicone of the hereinbelow indicated formula XII and 950 g of water with stirring and the mixture is converted into an emulsion in a conventional manner, for example using a slot homogenizer.

The thin liquid emulsion includes 10% by weight of the emulsifier according to the invention. It possesses very good stability in storage (stable for more than 60 days at 23° C., 40° C. and 50° C.) and is very easily thinnable with water to use concentration. It is stable to hard water to at least 40° German hardness.

Oxo thick oils 911 and 13 can be reacted and converted into formulations in a similar manner.

The silicone used in this example has the following formula XII:

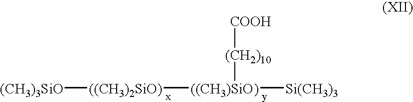

where the sum total of x and y is about 140-150 and y is about 3.

Properties of formulations according to the invention

The formulations obtained according to Examples 1 to 4, as reported above, have very good stability in storage and are readily thinnable with water to use concentration.

Even a formulation according to the invention prepared according to Example 4 with only 5% by weight of the emulsifier according to the invention has a stability of above 60 days in storage at 23° C., 40° C. and 60° C.

For comparison, a formulation was prepared according to part B of Example 4 using, instead of the emulsifier according to the invention, 10% by weight of N-oleoylsarcosine, a commercially available emulsifier which is very effective and therefore widely used in the hydrophobicizer field despite its relatively high cost. This formulation exhibited a substantially lower stability in storage. When stored at 23° C. an aqueous separation occurred after just 6 days (20% of the emulsion are relatively clear).

Only a formulation with 15% by weight of this commercially available emulsifier remained stable for more than 14 days at 23° C.

Just comparing the quantities of emulsifier needed to stabilize the formulations shows that only about the threefold amount of the sarcosine emulsifier reaches approximately the effectiveness of the emulsifier system according to the invention.

Considering in addition the cost-benefit ratio, the emulsifier according to the invention is found to have an at least tenfold superiority compared with the good commercially available product.

### APPLICATION EXAMPLES

### EXAMPLE 6

A chrome-tanned (wet blue) cattle hide 1.8-2.0 mm in shaved thickness, which had been deacidified to a pH of 5.0, was drummed for

30 min with 2% by weight of a commercially available polytan and then for

60 min with 3% by weight of a commercially available mimosa extract,

3% by weight of a commercially available resin tanning agent based on melamine condensation products and

1% by weight of a commercially available retanning agent based on phenol condensation products and then for

60 min with 2% by weight of a commercially available leather dye, the percentages being based on the shaved weight.

The leather was subsequently drummed for 90 min with 10% by weight of a formulation prepared according to Example 1, based on shaved weight, and the float was adjusted with formic acid to a pH of about 3.6 to 3.8, and thereafter the leather was washed. This was followed for 90 min by a mineral salt fixation with 3% by weight of a commercially available chrome tannin in the drum.

The leather was then washed, mechanically set out and dried.

The leather obtained was soft, supple, possessed a pleasant hand and a uniform color.

The Bally penetrometer test at 15% compression yielded the following results:

Water penetration: after 60 min,

Dynamic water absorption: 30% by weight after 6 hours.

### EXAMPLE 7

Example 6 is repeated except that the same amount of the formulation of Example 5 (formulation with addition of silicone) is used instead of the formulation of Example 1.

The Bally penetrometer test at 15% compression yielded the following results:

No water penetration after 24 hours,

Dynamic water absorption: 22% by weight after 24 hours.

We claim:

1. Mixtures of monoesters of di- or tribasic carboxylic acids of the formulae I and II
(MOOC)a-R1-CO—OR2 (I)
[(MOOC)a-R1-CO—O]2R3 (II)
where a is 1 or 2, M is hydrogen or one metal equivalent,
R1 is a di- or trivalent saturated or mono- or diunsaturated aliphatic or cycloaliphatic hydrocarbon radical of 2 to 6 carbon atoms with or without sulfonic acid group substitution or is a di- or trivalent aromatic hydrocarbon radical of 6 carbon atoms,
R2 is predominantly branched unsubstituted or hydroxyl-, alkoxy-, alkylcarbonyloxy- or alkoxycarbonyl-substituted alkyl of 9 to 51 carbon atoms, and
R3 is alkanediyl of 10 to 30 carbon atoms,
with OH-free ethers and esters of 18 to 45 carbon atoms and optionally alkanols of the formula R2OH and alkanediols of the formula R3(OH)2, the fraction of —OR2 groups being up to 85%, the fraction of (—O)2R3 groups being up to 16% and the fraction of OH-free ethers and esters being up to 45%, based on the sum total SUG of the weights of the OH-free ethers, OH-free esters, alkanols, alkanediols and —OR2- and (—O)2R3 groups present in the mixture.

2. Mixtures as claimed in claim 1, wherein
R1 is a divalent saturated aliphatic hydrocarbon radical of 2 to 4 carbon atoms with or without sulfonic acid group substitution
or is a divalent monounsaturated aliphatic hydrocarbon radical of 2 to 4 carbon atoms,
or is a divalent or trivalent saturated cycloaliphatic hydrocarbon radical of 6 carbon atoms with or without sulfonic acid group substitution
or is a di- or trivalent cycloaliphatic hydrocarbon radical of 6 carbon atoms having one or if appropriate two double bonds
or is a di- or trivalent aromatic hydrocarbon radical of 6 carbon atoms,
R2 comprises the radicals R2A, R2B, R2C, R2D and R2E, where
R2A denotes 1-alkyl and 2-alkyl radicals of 9 to 15 carbon atoms having an average molecular weight of MA
R2B denotes 2-alkyl-1-alkyl radicals of 18 to 30 carbon atoms having an average molecular weight of MB
R2C denotes x-alkyl-y-alkyl radicals of 18 to 30 carbon atoms having an average molecular weight of MC
R2D denotes 1-alkoxyalkyl radicals of 18 to 30 carbon atoms having an average molecular weight of MD
R2E denotes x-alkylcarbonyloxy-y-alkyl radicals and/or x-alkoxycarbonyl-y-alkyl radicals of 27 to 51 carbon atoms having an average molecular weight of ME,
R3 comprises the radicals R3F and R3G, where
R3F denotes 1,2-alkanediyl and/or 2-alkyl-1,3-alkanediyl of 10 to 16 carbon atoms having an average molecular weight of MF
R3G denotes 1,3-alkylalkanediyl of 18 to 30 carbon atoms having an average molecular weight of MG,
with OH-free ethers and esters of 18 to 45 carbon atoms and optionally alkanols of the formula R2OH and alkanediols of the formula R3(OH)2,
the —OR2A groups having a fraction of A[%]=0 to 20% by weight,
the —OR2B groups having a fraction of B[%]=0 to 40% by weight,
the —OR2C groups having a fraction of C[%]=0 to 10% by weight,
the —OR2D groups having a fraction of D[%]=0 to 20% by weight,
the —OR2E groups having a fraction of E[%]=0 to 25% by weight,
the (—O)2R3F- groups having a fraction of F[%]=0 to 7.5% by weight,
the (—O)2R3G- groups having a fraction of G[%]=0 to 8% by weight,
and the OH-free ethers and esters having a fraction of H[%} =0 to 45% by weight,
based on the sum total SUG of the weights of the OH-free ethers and esters, alkanols, alkanediols and OR2 and (—O)2R3 groups present in the mixture.

3. Mixtures as claimed in claims 1 and 2, wherein the fractions of the structures —OR2A, —OR2B, —OR2C, —OR2D, —OR2E, —OR3FO— and —OR3GO— present in the building groups —OR2 and —OR3 and the fraction of ethers and esters are selected in such a way within the framework of the above-indicated limits that the number OHZ defined by equation (GL1)
$$OHZ = 561 \cdot ( \frac{A [ \% ]}{MA} + \frac{B [ \% ]}{MB} + \frac{C [ \% ]}{MC} + \frac{D [ \% ]}{MD} + \frac{E [ \% ]}{ME} ) + 1120 \cdot ( \frac{F [ \% ]}{MF} + \frac{G [ \% ]}{MG} ) . \qquad ( GL1 )$$
where A[%], B[%], C[%], D[%], E[%], F[%] and G[%] are the abovementioned percentages of the structures present in the building groups —OR2 and —OR3, based on the sum total weight SUG, and MA, MB, MC, MD, ME, MF and MG are the molecular weights of said structures, is in the range from 65 to 160 and preferably in the range from 90 to 140.

4. A process for preparing the mixtures of monoesters of the formulae I and II of claim 1 by reacting inner anhydrides of di- or tribasic carboxylic acids with alkanols of medium and/or relatively large chain length, which comprises
A) reacting inner anhydrides of the formula IV 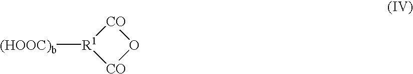
where b is 0 or 1, and
R1 is a di- or trivalent saturated or mono- or diunsaturated aliphatic or cycloaliphatic hydrocarbon radical of 2 to 6 carbon atoms or a di- or trivalent aromatic hydrocarbon radical of 6 carbon atoms, with or without a sulfo group,
at from about 20 to not more than 120° C. and preferably at from 75 to 100° C. in an equivalent ratio of from 1 to 1 to 1 to 5 with a mixture of
alcohols of the formula R2OH, where
R2 is predominantly branched, optionally hydroxyl-, alkoxy-, alkylcarbonyloxy- or alkoxycarbonyl-substituted alkyl of 9 to 51 carbon atoms,
alkanediols of the formula (HO)2R3, where
R3 is alkanediyl of 10 to 30 carbon atoms, and OH-free ethers and esters of 18 to 45 carbon atoms, and
B) to prepare mixtures according to the invention in which R1 has a sulfo group from a sulfo-free anhydride (IV) conducting the preparation step A) using an anhydride (IV) whose R1 radical is at least monounsaturated and then reacting the monoester mixture obtained according to section A), if appropriate after at least partial neutralization of its free carboxyl groups, in a conventional manner with a water-soluble sulfite, bisulfite or disulfite to add the sulfite or bisulfite to the double bond of the R1 group to form the desired sulfonic acid.

5. A process as claimed in claim 4, wherein the inner anhydrides used have the formula IV where
R1 is a divalent saturated aliphatic hydrocarbon radical of 2 to 4 carbon atoms with or without sulfonic acid group substitution
or is a divalent monounsaturated aliphatic hydrocarbon radical of 2 to 4 carbon atoms,
or is a divalent or trivalent saturated cycloaliphatic hydrocarbon radical of 6 carbon atoms with or without sulfonic acid group substitution
or is a di- or trivalent cycloaliphatic hydrocarbon radical, which is saturated, of 6 carbon atoms or having one or if appropriate two double bonds
or is a di- or trivalent aromatic hydrocarbon radical of 6 carbon atoms.

6. A process as claimed in claims 4 and 5, using mixtures of alcohols R2OH and diols R3(OH)2 comprising
from 0 to 20% by weight and preferably from 5 to 15% by weight of C9-C15 alkanols
from 0 to 40% by weight and preferably from 20 to 35% by weight of C18-C30 2-alkyl alcohols
from 0 to 10% by weight and preferably from 1 to 5% by weight of C18-C30 secondary alcohols
from 0 to 20% by weight and preferably from 5 to 15% by weight of C18-C30 ether alcohols
from 0 to 25% by weight and preferably from 10 to 20% by weight of C27-C51 ester alcohols
from 0 to 7.5% by weight and preferably from 0.8 to 5% by weight of C10-C16 1,2-diols
from 0 to 8% by weight and preferably from 1 to 5% by weight of C18-C30 diols and
from 0 to 45% by weight and preferably from 10 to 40% by weight of OH-free C18-C45 ethers and esters.

7. A process as claimed in claims 4 to 6, wherein the inner anhydrides of the formula IV are reacted with oxo thick oils as an alcohol component.

8. A process as claimed in claims 4 to 7, wherein the inner anhydrides of formula IV are reacted with oxo thick oils of the types “oxo oil 911 ”, “oxo oil 13” and/or “oxo oil 135”.

9. The use of the monoester mixtures of claim 1 as emulsifiers after partial or complete neutralization of the carboxylic acid groups.

10. The use of the monoester mixtures of claim 1 as assistants in leather manufacture.

11. A use as claimed in claim 9, wherein the monoester mixtures of claim 1 are used in combination with leather fatliquoring agents and/or leather hydrophobicizing agents.

12. Formulations including fatliquoring and/or hydrophobicizing substances required for fatliquoring and/or hydrophobicizing leather as well as an effective fraction of the monoester mixtures of claim 1.
